SEQUENCE LISTING (15 sequences)

SEQ ID NO: 1 - DNA, 16 bp, Artificial Sequence
  Description of Artificial SequencePCR primer
aagcttttttttttta

SEQ ID NO: 2 - DNA, 16 bp, Artificial Sequence
  Description of Artificial SequencePCR primer
aagctttttttttttc

SEQ ID NO: 3 - DNA, 16 bp, Artificial Sequence
  Description of Artificial SequencePCR primer
aagctttttttttttg

SEQ ID NO: 4 - DNA, 13 bp, Artificial Sequence
  Description of Artificial SequencePCR primer
aagcttgattgcc

SEQ ID NO: 5 - DNA, 13 bp, Artificial Sequence
  Description of Artificial SequencePCR primer
aagcttcgactgt

SEQ ID NO: 6 - DNA, 13 bp, Artificial Sequence
  Description of Artificial SequencePCR primer
aagctttggtcag

SEQ ID NO: 7 - DNA, 13 bp, Artificial Sequence
  Description of Artificial SequencePCR primer
aagcttctcaacg

SEQ ID NO: 8 - DNA, 13 bp, Artificial Sequence
  Description of Artificial SequencePCR primer
aagcttagtaggc

SEQ ID NO: 9 - DNA, 13 bp, Artificial Sequence
  Description of Artificial SequencePCR primer
aagcttgcaccat

SEQ ID NO: 10 - DNA, 13 bp, Artificial Sequence
  Description of Artificial SequencePCR primer
aagcttaacgagg

SEQ ID NO: 11 - DNA, 13 bp, Artificial Sequence
  Description of Artificial SequencePCR primer
aagcttttaccgc

SEQ ID NO: 12 - DNA, 13 bp, Artificial Sequence
  Description of Artificial SequencePCR primer
aagcttcattccg

SEQ ID NO: 13 - DNA, 13 bp, Artificial Sequence
  Description of Artificial SequencePCR primer
aagcttccacgta

SEQ ID NO: 14 - DNA, 17 bp, Artificial Sequence
  Description of Artificial SequencePCR primer
gacgcgaacgaagcaac

SEQ ID NO: 15 - DNA, 17 bp, Artificial Sequence
  Description of Artificial SequencePCR primer
cgacaacaccgataatc

(19) United States
(12) Patent Application Publication
(10) Pub. No.: US 2004/0014636 A1
(43) Pub. Date: Jan. 22, 2004

(54) Pharmaceutical compositions comprising a modulator of adamts-1

(76) Inventors: Peter Brodin, Molndal (SE); Anders Thelin, Molndal (SE)

Correspondence Address: ROPES & GRAY LLP, ONE INTERNATIONAL PLACE, BOSTON, MA, 02110-2624, US

(21) Appl. No.: 10/362,262

(22) PCT Filed: Aug. 16, 2001
(86) PCT No.: PCT/GB01/03650

(30) Foreign Application Priority Data
Aug. 22, 2000 (SE) 0002973-6

(51) Int. Cl. A61K 31/00; G01N 33/53; G01N 33/567

(52) U.S. Cl. 514/1; 435/7.2

(57) ABSTRACT

The present invention is based on the discovery that the metalloproteinase, ADAMTS-1 (A Disintegrin And Metalloproteinase), is associated with obesity, atherosclerosis, insulin resistance syndrome and non-insulin dependent diabetes. The application is directed to methods for screening for specidic modulators of ADAMS-1 activity, and the use of said modulators for treating the above-mentioned diseases.

The present invention is based on the discovery that the metalloproteinase, ADAMTS-1 (A Disintegrin And Metalloproteinase), is associated with obesity, atherosclerosis, insulin resistance syndrome and non-insulin dependent diabetes.

The ADAM (A Disintegrin And Metalloproteinase) family of metalloproteinases, containing 30 members to date, have been identified in organisms ranging from yeast to humans (Wolfsberg et al., 1998; Blobel, 1997; Tang, 2001). They have conserved domain structures. ADAMs have been implicated in diverse biological processes, such as shedding of cell surface molecules and adhesion to cells and matrix proteins. For example, ADAM 17 (TACE/TNFα-convertase) cleaves and releases the membrane bound form of TNFα; the Drosophila enzyme kuzbanian and its mammalian homologue (ADAM 10) have been shown to cleave the extracellular domain of the transmembrane receptor Notch. ADAMs 1 and 2 (fertilin α and β) have been shown to be essential for sperm-egg fusion during fertilization. They have also been shown to be potential players in pathological events such as cancer metastasis and inflammation.

Within the past 4 years, a new subset of ADAM-related proteins, known as ADAMTS (A Disintegrin-like And Metalloprotease with Thrombospondin type 1 motif), have been identified; there are about 10 known members to date, half of which have no known function. The ADAMTS' differ from the previously known ADAMs by the lack of the transmembrane domain and the presence of variable numbers of thrombospondin type I (ITSP-1) repeats. The fist member, ADAMTS-1, was cloned from a mouse cachexic colon subline and shown to be inducible by IL-1, suggesting a role as an inflammation associated gene (Juno et al., 1997). It was also found up-regulated in the kidney and heart by intravenous administration of lipopolysaccharide (LPS) in mice, again suggesting the gene may be induced during inflammatory responses (Kuno et al., 1997; 1998). The protein is secreted and binds to extracellular matrix and heparin through the thrombospondin and spacer domains (Kuno et al., 1999). Human ADAMTS-1 was identified by a separate group searching for proteins containing the anti-angiogenic type 1 repeats of thrombopsondin-1, which they called METH-1; the TSP-1 repeats were shown to be required for the potent anti-angiogenic properties observed (Vazquez et al., 1999). METH-1 has also been described in WO 99/37660 and WO 00/71577 (Irulea-Arispe et al). Phylogenetically, ADAMTS-1 has the most homology to ADAMTS-4 and ADAMTS-8.

ADAMTS-1-deficient mice have been generated; they are viable but exhibit growth retardation, impaired female fertility, and defects in the kidney (Shindo et al., 2000). The kidney defect is consistent with the high levels of expression in the embryonic kidney of normal mice; however, the message levels are significantly reduced in the adult (MRC biotechnology; Vazquez, 1999). Gon-1, a C. elegans ADAMTS family member, mutants have been generated; they displayed severe defects in gonad development (Blellock and Kimble, 1999). The distinct phenotypes observed in ADAMTS-deficient mouse and worm suggest that at least some members of this family have some specific and nonredundant roles in cell migration/remodelling during development. This is in contrast to that observed for deficiencies by many metalloproteases, which show surprisingly mild phenotypes. The effects of the ADAMTS-1 ^{-/-} genotype in obesity, IRS, NIDDM or atherosclerosis were not investigated.

EP 874 050 (SmithKline Beecham/Human Genome Science) concerns the human analogue to the mouse ADAMTS-1, in the application designated integrin ligand ITGL-TSP. The indications mentioned to be related to ADAMTS-1 are limited to angiogenic diseases (cancer, cancer metastasis, chronic inflammatory disorders, rheumatoid arthritis, atherosclerosis, macular degeneration, diabetic retinopathy), restenosis, Alzheimer's disease and tissue remodelling.

WO 98/55643 (Kureha Chemical Industry) from the Kuno group covers human ADAMTS-1 protein, and its use as an agent for decreasing the leukocyte and thrombocyte blood count and increasing the erythrocyte blood count, e.g. for treatment of inflammatory diseases such as rheumatoid arthritis, hepatitis, nephritis, Crohn's disease, asthma and ARDS.

Recently ADAMTS-1 ^{-/-} mice have been generated by gene targeting. These mice demonstrate a renal phenotype resembling the human ureteropelvic junction obstruction. The effects of the ADAMTS-1 ^{-/-} genotype in obesity, IRS, NIDDM or atheroscleorosis were not investigated.

Presently the degree of information known about the role of ADAMTS-1 in disease has been limited and generally speculative. Therefore is a need for a better understanding of the specific function of ADAMTS-1 in disease. Furthermore, there is a need for a better understanding of the nature of the underlying physiology of the important diseases of obesity, atherosclerosis, IRS and NIDDM. None of the known art mentions the connection between the specific level of ADAMTS-1 per se expression and obesity, atherosclerosis or IRS, or the possibility to prevent or treat these conditions by specifically modulating the expression level or the activity of ADAMTS-1.

The present invention is based on the discovery that ADAMTS-1 is specifically associated with obesity, atherosclerosis, insulin resistance syndrome and non-insulin dependent diabetes.

According to one aspect of the present invention there is provided use of a compound able to modulate specifically the activity or amount of ADAMTS-1 in preparation of a medicament for the treatment of a disease indelendently selected from obesity, IRS, NIDDM or atherosclerosis. A preferred use is of a compound able to reduce specifically the activity or amount of ADAMTS-1 in preparation of a medicament for the treatment of obesity, IRS, NIDDM or atherosclerosis. In another embodiment, a preferred use is of a compound able to increase specifically the activity or amount of ADAMTS-1 in preparation of a medicament for the treatment of obesity, IRS, NIDDM or atherosclerosis. Another embodiment of the invention is use of a compound able to reduce specifically the activity of ADAMTS-1 in preparation of a medicament for the treatment of obesity, IRS, NIDDM or atherosclerosis. Another embodiment of the invention is of a compound able to increase specifically the activity of ADAMTS-1 in preparation of a medicament for the treatment of obesity, IRS, NIDDM or atherosclerosis.

The term “a compound able to modulate specifically the activity or amount of ADAMTS-1” means that the principal pharmaceutical activity relating to obesity, IRS, NIDDM or atherosclerosis of the compound is dependent on its effect on ADAMTS-1. For example, thiazolidinone compounds such as for example rosiglitazone, fall outside the scope of this definition because they have significant pharmaceutical activity through PPAR-γ, see Willson et al (2000), J Med Chem, 43, 527-550.

In particular, compounds able to modulate specifically the amount of ADAMTS-1 refers to compounds that modulate the amount of ADAMTS-1 through a direct effect on the ADAMTS-1 gene or its expression; the ADAMTS-1 mRNA, its turn-over, processing, degradation or stability; or the ADAMTS-1 protein, its turnover, processing, degradation, or stability.

In particular, compounds able to modulate specifically the activity of ADAMTS-1 refers to compounds that modulate the activity of ADAMTS-1 without significantly modulating the activity of ADAM 17 (TNF-□ converting enzyme, TACE), MMP-1 (interstitial collagenase), MMP-14 (membrane type 1-matrix metalloproteinase), MMP-19 (rheumatoid associated arthritis-associated MMP) and PPAR. However, it is contemplated that compounds having effects on some of the other ADAMTSs, such as for example the aggrecanases ADAMTS-4 or ADAMTS-5 would fall within the definition. Without wishing to be bound by theoretical considerations, it may even be beneficial to have an effect on some other proteins e.g. the aggrecanases.

The activity of a compound at ADAMTS-1 per se may be measured through a direct effect on the ADAMTS-1 enzyme activity as measured by the enzyme assays exemplified herein.

According to another aspect of the present invention there is provided a method of screening for a compound potentially useful for treatment of obesity, IRS, NIDDM or atherosclerosis which comprises assay of the compound for its ability to modulate specifically the activity or amount ADAMTS-1. Preferably the assay is indelendently selected from:

i) measurement of ADAMTS-1 activity using a cell line which expresses ADAMTS-1 or using purified ADAMTS-1 protein; and

ii) measurement of ADAMTS-1 transcription or translation in a cell line expressing ADAMTS-1. Preferably the cell line is a mouse 3T3-L1 cell. Preferably the protein is human recombinant ADAMTS-1.

The amino acid sequence of human ADAMTS-1 can e.g. be obtained from the SwissProt database as id ATS1_HUMAN, DNA sequences encoding human ADAMTS-1 can be e.g. obtained from the EMBL database as accession nos. AP170084, AF060152, AP207664, and AP001697. The amino acid sequence of mouse ADAMTS-1 can e.g. be obtained from the SwissProt database as id ATS1_MOUSE, DNA sequences encoding mouse ADAMTS-1 can be e.g obtained from the EMBL database as accesion nos. AB001735 and D67076.

According to another aspect of the present invention there is provided a method of of preparing a pharmaceutical composition which comprises:

i) identifying a compound as useful for treatment of obesity, IRS, NIDDM or atherosclerosis according to a method as described herein; and

ii) mixing the compound or a pharmaceutically acceptable salt thereof with a pharmaceutically acceptable excipient or diluent.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound will naturally vary according to the nature and severity of the conditions, the age and sex of the animal or patient and the route of administration, according to well known principles of medicine.

In using a compound for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

The invention is further described in the non-limiting Examples below with reference to the following drawings in which:

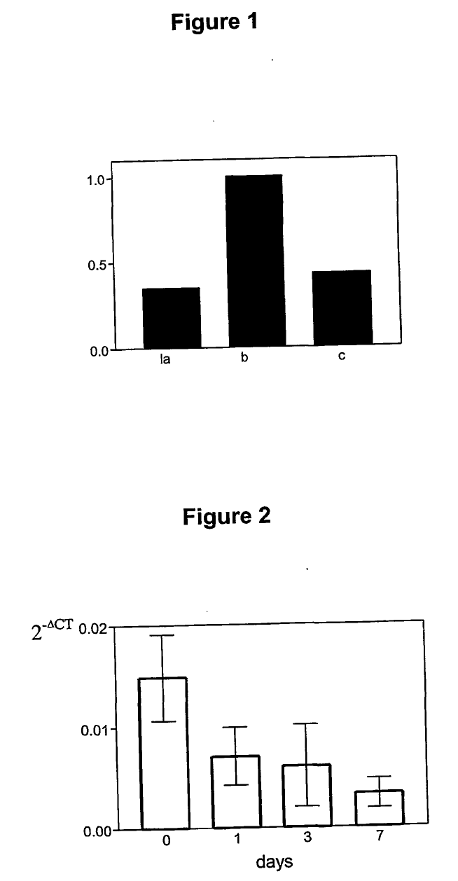

FIG. 1. Real time PCR comparing lean mice, ob/ob mice, and ob/ob mice treated with rosiglitazone.

A comparison of ADAMTS-1 expression in mesenterial fat (5 animals per group) from lean (1a), untreated ob/ob mice (b) & ob/ob mice treated with Rosiglitazone for 7 days (c). ADAMTS-1 mRNA levels are significantly elevated in obese (ob/ob) (a) compared with lean (−/ob) animals (b). Treatment with Rosiglitazone decreases the expression in obese mice close to levels found in lean animals (c).

FIG. 2. Real time PCR analysis of epididymal fat of mice treated with rosiglitazone.

A time course study (3 animals per group) treated with Rosiglitazone daily. Real time PCR quantitation of ADAMTS-1 expression in epididymal fat was performed at 0, 1 3,& 7 days. Expression levels decrease substantially in epididymal fat after the first Rosiglitazone administration to ob/ob mice, and is further reduced over a 7 day period. The downregulation of ADAMTS-1 precedes the effects on plasma glucose and triglycerides which are not lowered by the first administration of Rosiglitazone.

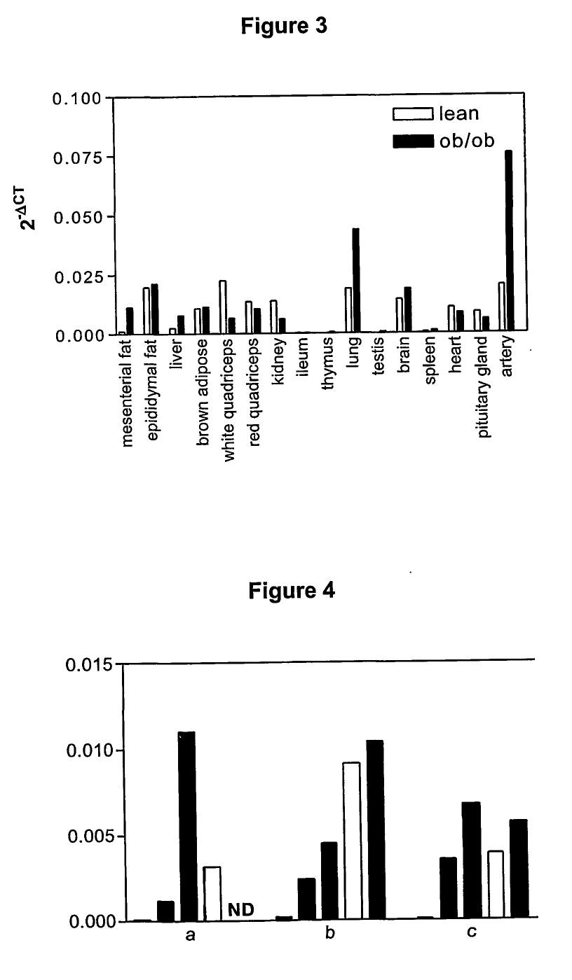

FIG. 3. Real time PCR analysis of various tissues.

Analysis of expression levels of ADAMTS-1 comparing message levels in lean compared to ob/ob nmice shows the tissue distribution of mouse ADAMTS-1. Real time PCR quantitation on pooled cDNA from 3 animals was normalised against internal control (ribosomal protein 36B4). Expression is up-regulated in several tissues in obese animals. Besides mesenterial fat, the up-regulation is most pronounced in liver, lung and notably in aorta

FIG. 4. ADAMTS-1 message levels in various tissues in treated mice.

ADAMTS-1 message levels in various tissues in mice treated with rosiglitazone for 7 days shows a comparison of ADAMTS-1 expression in various tissues (5 animals per group) from lean (a), untreated ob/ob mice (b) & ob/ob mice treated with Rosiglitazone for 7 days (c). The tissues for each group were as follows, in left to right order bone marrow; liver, quadriceps; white adipose; and brown adipose.

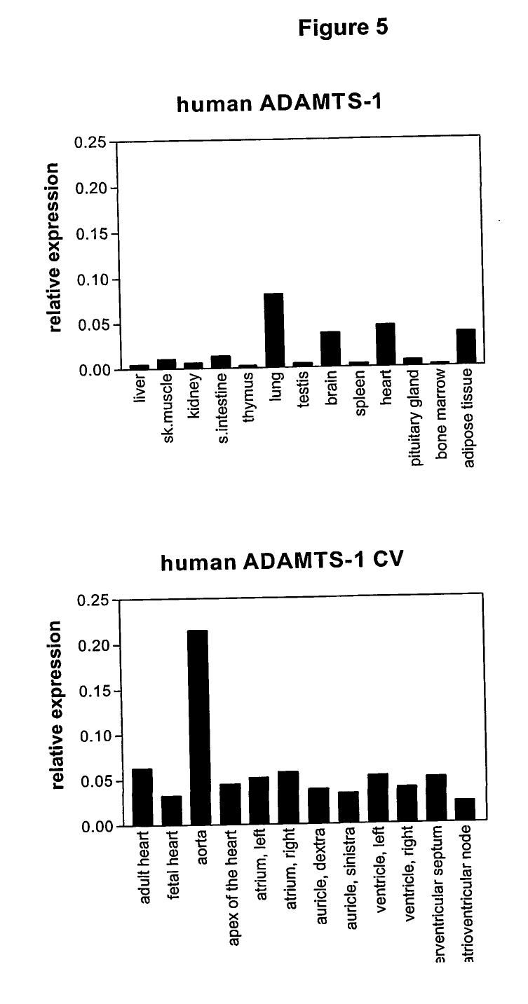

FIG. 5. ADAMTS-1 levels in various human tissues

Real time PCR quantitation on pooled cDNA from several individuals was normalised against internal control (ribosomal protein 36B4) shows the tissue distribution of human ADAMTS-1. ADAMTS-1 expression is significantly higher in the heart than in most other tissues, particularly in the aorta.

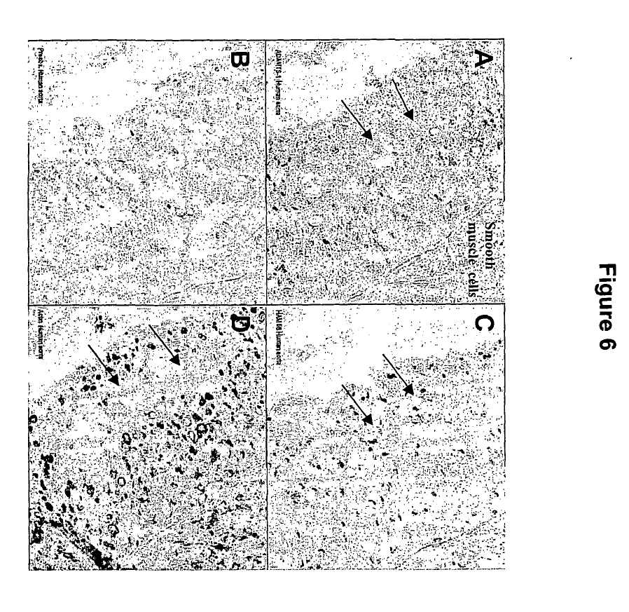

FIG. 6. Immunohistochemistry of Type I aortic lesion.

Immunohistochemistry with antibodies against ADAMTS-1, α-actin, and macrophage in early fatty streak (type I lesion). A. ADAMTS-1 labelling is seen in foam-like cells (arrow) and smooth muscle cells. B. Preabsorption control. C. Macrophage-like immunoreactivity (RAM-56). Some staining colocalize with ADAMTS-1 staining (arrows). D. Smooth muscle labelling (□-actin).

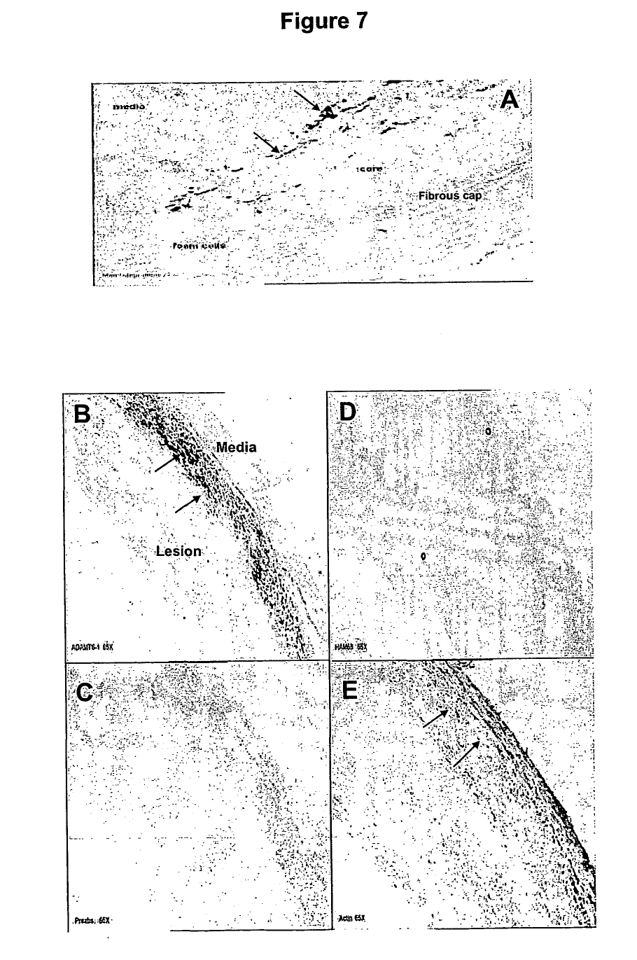

FIG. 7. Immunohistochemistry.

Immunohistochemistry with antibodies against human ADAMTS-1, α-actin, and macrophage in an advanced plaque (type III-IV) in the aorta (A) or coronary artery (B-E). A. ADAMTS-1 like immunoreactivity is seen in the matrix-like core at the base of the aortic plaque (arrow). B. ADAMTS-1 like immunoreactivity at the base of the plaque, close to the media (arrows). C. Preabsorbed antibody control for ADAMTS-1 staining. D. Macrophage (HAM-56) labelling. E. Actin-like immunoreactivity is found in the smooth muscle cells of the media and basally in the plaque. Arrows indicate staining co-localize with ADAMTS-1.

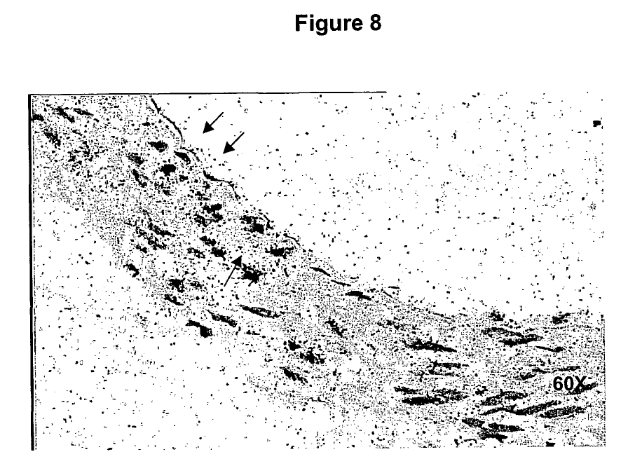

FIG. 8. In situ hybridization.

In situ hybridization for ADAMTS-1 message in the aorta of ApoE/LDL Receptor—deficient mouse. Note staining is observed both in endothelial and smooth muscle cell layers (arrows). 60×.

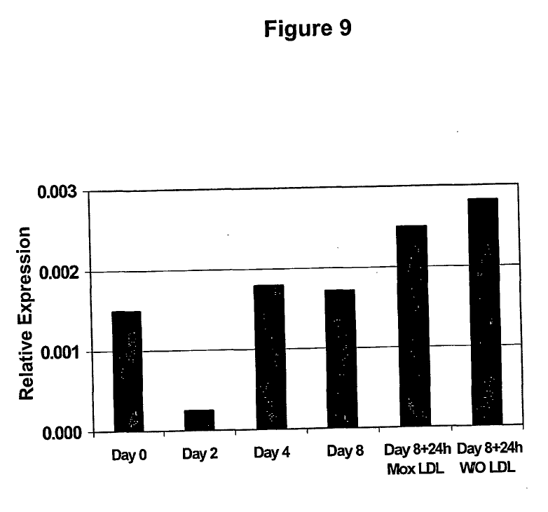

FIG. 9. Expression of hADAMTS-1 in ThP-1 cells.

Real time PCR analysis for ADAMTS-1 message levels in THP-1 cells treated with PMA at day 0, 2, 4, 8, 8+24 hours, (8+24 hours with mildly oxidized LDL).

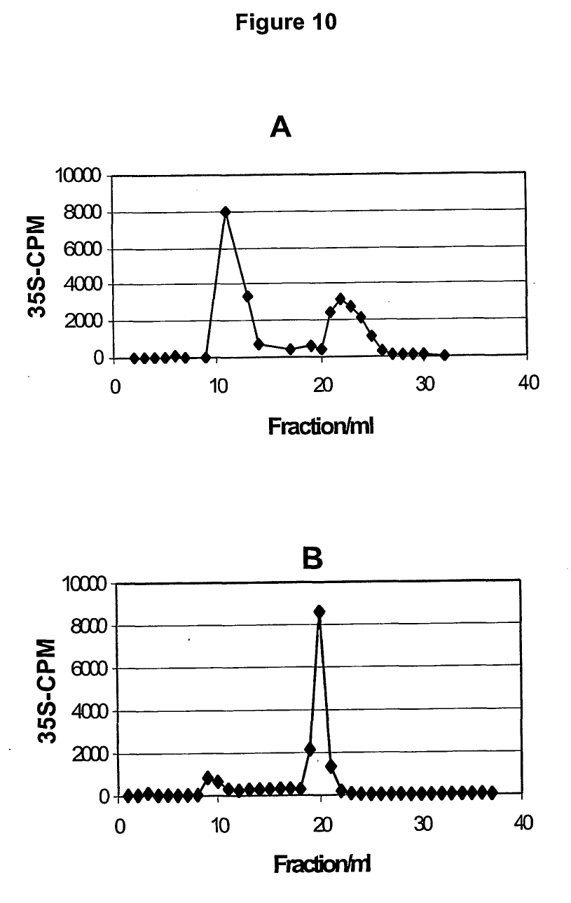

FIG. 10. Size exclusion chromatography of proteoglycan from ASMCs.

A. Analysis of total proteoglycan secreted by primary aortic smooth muscle cells cultured with 35S and 3H. The two proteoglycan population can be separated according to size by size exclusion chromatography. The large proteoglycan population is made up of primarily versican. B. When the total proteoglycan population is incubated with ADAMTS-1 prior to separation by size exclusion chromatography, the size of the large proteoglycan peak is diminished and the size of the smaller proteoglycan population is increased

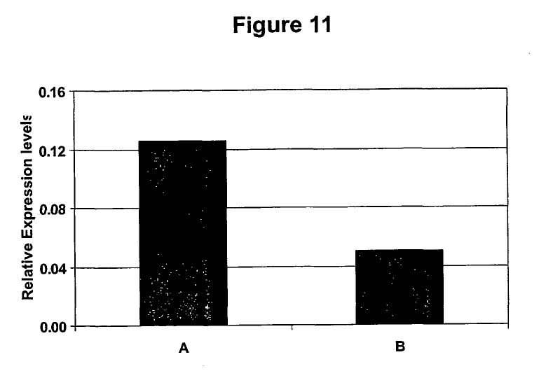

FIG. 11. Real time PCR analysis of aortic SMCs.

Real time PCR analysis for ADAMTS-1 message levels in A) proliferating primary human aortic smooth muscle cells compared to B) resting confluent human aortic smooth muscle cells.

### EXAMPLE 1

Role of ADAMTS-1 in IRS, NIDDM, Obesity and Atherosclerosis

1.1 Materials and Methods

Primers

| | |
|---|---|
| H-T₁₁-A | SEQ ID NO: 1 |
| H-T₁₁-C | SEQ ID NO: 2 |
| H-T₁₁-G | SEQ ID NO: 3 |
| H-AP-1 | SEQ ID NO: 4 |
| H-AP-2 | SEQ ID NO: 5 |
| H-AP-3 | SEQ ID NO: 6 |
| H-AP-4 | SEQ ID NO: 7 |
| H-AP-5 | SEQ ID NO: 8 |
| H-AP-6 | SEQ ID NO: 9 |
| H-AP-7 | SEQ ID NO: 10 |
| H-AP-8 | SEQ ID NO: 11 |
| H-AP-9 | SEQ ID NO: 12 |
| H-AP-10 | SEQ ID NO: 13 |
| Rgh | SEQ ID NO: 14 |
| Lgh | SEQ ID NO: 15 |

Animals, cell culture and treatment. Nine weeks old ob/ob mice were treated for seven days with Rosiglitazone at 30 μmol/kg/day. The drug was administrated orally using a gavage. Control animals were fed vehicle (0.1% DMSO). To reduce variation in genetic background male sibling pairs were used with one being treated with drug and the other with vehicle. The animals had free access to water and normal mice chow. 3T3-L1 cells were grown in 175 cm² flasks to confluency. Dexamethasone at 2 μg/mL and methylisobutyl-xantine at 0.5 μM were then included in the media. This treatment was continued for two weeks. This will drive the differentiation of the cells to adipocytes. The dexamethasone/methylisobutyl-xantine were removed and cells were thereafter treated with Rosiglitazone at 1 μM for 24 hours. Control cells were also treated with dexamethasone/methylisobutyl-xantine but with vehicle instead of Rosiglitazone.

Tissue isolation and RNA extraction. From treated and control mice liver, mesenterial fat, epididimus fat, brown fat, white fibers from quadriceps (quadri/white), red fibers from quadriceps (quadri/red) and heart were isolated. Care was taken to remove contaminating tissues, blood and hair. All tissues were removed and snap-frozen in liquid nitrogen within 2 minutes after the animal was killed. Tissues were weighed and RNASTAT-60 (AMS Biotechnology) added. Tissues were homogenized with a Turrax-blender for one minute on ice. Total RNA was extracted according to suppliers protocol. Briefly, for tissue amounts up to 100 mg, 1 mL of extraction media was added and the tissue homogenized. The organic and water phase were separated by a centrifugation. The upper, water, phase, was isolated and RNA precipitated with one volume of isopropanol. RNA pellet was washed with 75% ice-cold ethanol. RNA pellet was dried and dissolved in DEPC treated water. For RNA extraction of 3T3-L1 cells the incubation media was poured off and RNASTAT-60 added. RNA was extracted as described above. To remove residual DNA the total RNA preparation was treated with DNAse. 50 μg RNA were incubated at 37° C. with 5 U DNAse (RQ1 DNAse, Promega) in 10 mM CaCl₂; 6 mM MgCl₂; 10 mM NaCl and 40 mM Tris-Cl pH 7.9 in a final volume of 100 μL. After 15 minutes the reaction was stopped by adding 4 μL 0.5 M EDTA. Protein was removed with a phenol/chloroform/isoamylalcohol extraction. RNA was ethanol precipitated, re-dissolved in DEPC treated water and quantitated by an OD reading at 260 nm. The quality of the RNA was also checked on an 1% agarose gel.

Differential display. The differential display was performed using reagents from GeneHunter Corp. (Nashville, Tenn.) (Liang and Pardee, 1992). In three parallel reactions total RNA was reverse transcribed using three different anchored primers, H-T11-A, H-T11-G and H-T11-C. Reactions were performed in duplicate. 0.2 μg of total RNA in 13.4 μL water were mixed with 1.6 μL 250 μM dNTP; 4 μL 5× RT buffer (125 nmM Tris-Cl pH 8.3, 188 mM KCl, 7.5 mM MgCl₂, 25 mM DTT) and 2 μL 2 μM anchored primer. Samples were incubated in a thermocycler, 65° C. for 5 min., 37° C. for 60 min. and 75° C. for 5 min. After five minutes at 37° C. M V reverse transcriptase was added. PCRs were set up using the cDNA produced and combinations of the three anchored primers and ten different random primers. For each primer combination the following reaction was set up. 9.2 μL water, 2 μL 10× PCR buffer (100 mM Tris-Cl pH 8.4, 500 mM KCl, 15 mM MgCl2 and 0.01% gelatin); 1.6 μL 25 μM dNTP; 2 μL 2 μM anchored primer, 2 μL RT reaction mix; 0.25 μL α-[33P]dATP, 2000 Ci/mmol and 0.2 μL AmpliTaq (Perkin-Elmer). Samples were incubated in a thermocycler, using the following temperature cycle. 1. 94° C. for 30 sec., 2. 40° C. for 2 min., 3. 72° C. for 30 sec., 4. goto step 1, 40 cycles and 5. 72° C. for 5 min. After the amplification 3.5 μL of the PCR reaction were mixed with 2 μL loading dye (95% formamide, 10 mM EDTA pH 8.0, 0.09% Xylene cyanole FF and 0.09% bromphenol blue). Immediately before loading on 6% PAGE with urea the samples were denaturated at 80° C. PCRs with the same primer combination from treated and untreated tissues/cells were loaded side by side. When the slower migrating xylene dye reached the bottom of the gel the electrophoresis was stopped. The gel was transferred to a filter paper and dried in a vacuum gel dryer. Radioactivity was detected by placing the dried gel against a Hyperfilm MX™ (Amersham). Bands which appear in the duplicate from one tissue but not the other were isolated by cutting out the band from the dried gel with a scalpel. To make sure that the correct band was cut out a second autoradiography film was exposed to the dried gel. Isolated bands were boiled in 100 μL water for 15 min. The gel and filter paper were spun down and the supernatant transferred to a fresh tube. The isolated fragment were re-amplified using the same PCR protocol as described above with one change. The dNTP concentration in the re-amplification was 20 μM. PCR products were analyzed on 1% agarose. If the PCR gave a product of expected size the PCR reaction mixture was used for ligation of the PCR product into the pCRTRAP vector (GeneHunter). Five μL water was mixed with 2 μL linearized pCRTRAP; 1 μL 10× ligation buffer (GeneHunter); 2.5 μL PCR product and 0.5 μL T4 DNA ligase (100 U). The ligation reaction was incubated at 16° C. overnight. Ten μL of ligation reaction mixture were transformed into 100 μL GH-competent cells (GeneHunter). Bacteria were plated onto LB with tetracycline (20 μg/mL). Colonies which appeared after overnight incubation at 37° C. were collected and lysed at 95° C. for 10 min. in 50 μL lysis buffer (GeneHunter). The size of the insert was checked using PCR with a vector specific primer pair (Rgh, Lgh). 10.2 μL water were mixed with 2 μL 10× PCR buffer, 1.6 μL 250 μM dNTP; 2 μL 2 μM Lgh primer, 2 μL 2 μM Rgh primer, 0.2 μL AmpliTaq (1 U) and 2 μL colony lysate. PCR were performed at 1. 94° C. for 30 sec., 2. 52° C. for 40 sec., 3. 72° C. for 1 min., 4. goto 1, 40 cycles and 5. 72° C. for 5 min. PCR products was analyzed on 1.5% agarose. In general five positive colonies were used to inoculate 5 mL LB media with tetracycline. Cultures were incubated over night. Cells were spun down and the pellet used for a Wizard (Promega) plasmid miniprep.

DNA sequencing. Inserts were sequenced using the Rgh or Lgh primer with the Thermocycler kit for dye terminator cycle sequencing (Perkin Elmer).

1.2 Results

Performance of differential display. In the reverse transcription step, three different anchored primers were used in three independent reactions. These primers have a poly T portion which will hybridize to the poly A tail in the 3′ end of mRNA. The last base is either a C, G or an A. This procedure subdivides all poly A transcripts in three cDNA pools. For each of the cDNA pools PCRs were set up using ten different random primers. This procedure subdivides and amplifies the cDNA pool further. The primer combinations used in this study typically generate approximately 150 fragments/primer combination. Using three different anchored and ten different random primers a total of 4500 fragments have been generated for each tissue. In the literature it has been estimated that 15000 genes are expressed at any given moment in a cell (6). Using this estimation it can be assumed that ⅓ of the expressed genes in each tissue has been analyzed. This assumes that each gene will generate only one fragment, this may not always be the case. Of the analyzed fragments approximately 150 were detected and isolated as differentially expressed. In the individual tissues 7-23 fragments with differential expression were detected with a mean around 15. This indicates that approximately 0.1% of expressed genes were affected by the drug treatment. The highest number of differentially expressed fragments were found in brown adipose tissue followed by liver, epididimus fat, mesenterial fat, quadri/white, quadri/red and heart. 3T3-L1 cells were affected to the same extent as liver in mice. This indicates that of the tissues studied here brown fat is the most affected by the drug treatment while heart is the least. Liver and fat tissues are more affected than the muscle tissues.

Fragments, up-down regulated by treatment, found in several tissues. Following drug treatment the levels of expression were up-regulated for ⅔ of the fragments and down-regulated for the remaning ⅓. In all tissues studied both up- and down-regulated expression were observed. The highest relative up-regulation of expression was detected in brown fat while the highest relative down-regulation was detected in heart

Bioinformatics analysis. The sequences obtained from the differential display experiment were compared to sequences found in DNA databases. EMBL non-EST, EMBL EST and Patseq were searched using the blastn algorithm. Hits with P(N) values lower than 10-10 in the EMBL non-EST database were used to identify fragments. Hits from other mammals (than mouse) were used for identification only if the differential display fragment aligned to the coding part of that cDNA/gene. If no hits were obtained in the EMBL non-EST database the EMBL EST database was searched. Only hits from mouse with P(N) lower than 1×10¹⁰ were recorded. The patent DNA database PatSeq was searched for patented sequences. If no significant non-EST or EST were found the differential display fragment was designated unknown. Somewhat less than half of the fragments in this study returned known genes when analyzed against databases. One quarter was only identified as ESTs and one quarter as unknown. In all tissues and cells studied here were approximately the same proportion between known/EST and unknown observed.

2 Identification of ADAMTS-1 Role in IRS, NIDDM, Obeisty and Atherosclerosis.

ADAMTS-1 was initially identified in an expression profiling experiment performed in order to more fully understand the mechanisms of action of PPARγ agonists and to find new molecular targets useful for treatment of insulin resistance syndrome (IRS)/non-insulin dependent diabetes (NIDDM). The thiazolidinedione (TZD) class of compounds used as insulin-sensitizing drugs for treatment of non-insulin dependent diabetes are known to act as ligands for the Peroxisome Proliferator-Activated Receptor γ (PPARγ). A differential display analysis was performed using pair wise comparisons of various organs and tissues from control and rosiglitazone-treated (TZD X103, BRL49653, ARH036133) ob/ob mice. These mice are leptin-deficient, obese and develop a condition resembling NIDDM with age; some of these symptoms, such as dyslipidemia and obesity, are exhibited by patients who are statistically likely to develop atherosclerosis. The differential display analysis resulted in the identification of more than 100 primary sequences derived from known genes, ESTs and unknown genes. The identified sequences were run through a confirmation process using real time quantitative PCR in order to sort out the true up or down regulated genes. Also, confirmed hits were further validated in time-course and tissue distribution experiments. Confirmed sequences were taken for bioinformatics and literature studies. 12 potential targets (4 known genes, 4 ESTs and 4 previously unknown genes) were selected for further studies.

One of the differentially expressed sequences corresponded to the mouse ADAMTS-1 mRNA, which was significantly elevated in obese ob/ob mice compared to lean littermates in mesenterial fat (FIG. 3). ADAMTS-1 message was down-regulated after 7 days of Rosiglitazone treatment (administered daily, 30 μmol/kg/day) in epididymal fat tissue (FIG. 2). Real time PCR quantitation on tissues from another set of identically treated animals showed that the down-regulation occurred also in mesenterial (FIG. 1) and brown fat tissue (FIG. 4). Observe that the measurements were done on pooled cDNA from 5 animals, hence the lack of error bars.

Subsequently it was found that in humans, the level of ADAMTS-1 is higher in the heart compared to most other tissues, particularly in the aorta (FIG. 5). Since proteases play an integral role both in atherogenesis and for plaque stability by remodelling and degrading ECM proteins, ADAMTS-1 became a potentially interesting target for atherosclerosis.

Conclusions:

1. ADAMTS-1 expression is up-regulated in fat tissue and aorta of obese (ob/ob) mice, and down-regulated in muscle.

2. Treatment of ob/ob mice with PPARγ agonists normalizes the expression in fat tissue (and to some extent in muscle)

3. ADAMTS-1 shows a tissue distribution that is relevant from a NIDDM, obesity and atherosclerosis perspective.

4. The gene is expressed (and reacts to PPARγ agonists) in an available cell system (mouse 3T3-L1 cells).

5. ADAMTS-1 homologues are found in suitable model organisms (mouse, C. elegans and Drosophila).

6. ADAMTS-1 belongs to a family of proteases/integrin binding proteins found to be involved in a multitude of processes in several important diseases.

7. The protein is exported, and therefore potentially relatively easy to express and purify.

8. The ADAMTS-1 molecule has several functional domains (pro-, metalloproteinase-, integrin binding- and matrix binding domains) that are useful for drug targeting.

Therefore ADAMTS-1 per se has been demonstrated for the first time to be a specific drug target of interest for NIDDM/RS, atherosclerosis and obesity treatment. Without wishing to be bound by theoretical considerations, its roles might be in tissue/matrix remodelling, differentiation or the release/modification of cytokines, growth factors and receptors.

### EXAMPLE 2

Assay Development

The complete mouse ADAMTS-1 cDNA has been cloned from epididymal fat tissue and inserted in mammalian expression vectors (for both constitutive and inducible expression). The protein is expressed both in native form and with an epitope tag (FLAG) in the C-terminus in order to simplify detection and purification. The human ADAMTS-1 homologue is cloned and expressed analogously. Antibodies against various functional domains in the ADAMTS-1 molecule are contemplated.

Assays:

ADAMTS-1 protease activity (cell based or using purified recombinant protein)

ADAMTS-1 activation (cell based or using purified recombinant protein) WHAT

Activation of ADAMTS-1 transcription in suitable cell lines

Disintegrin assay for monitoring ADAMTS-1l/(target) protein interactions

Selection of synthetic substrate through peptide library technology

Assays for pro-domain cleavage/ADAMTS-1 activation

Further assay information is presented in Example 9 below.

### EXAMPLE 3

Pharmaceutical Compositions

The following illustrate representative pharmaceutical dosage forms capable of preparation through the method of the invention as defined herein (the active ingredient being termed “Compound X”), for therapeutic or prophylactic use in humans:

| (a) Tablet I | mg/tablet |
|---|---|
| Compound X | 100 |
| Lactose Ph.Eur | 182.75 |
| Croscarmellose sodium | 12.0 |
| Maize starch paste (5% w/v paste) | 2.25 |
| Magnesium stearate | 3.0 |
| (b) Tablet II | mg/tablet |
| Compound X | 50 |
| Lactose Ph.Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |
| (c) Tablet III | mg/tablet |
| Compound X | 1.0 |
| Lactose Ph.Eur | 93.25 |
| Croscarmellose sodium | 4.0 |
| Maize starch paste (5% w/v paste) | 0.75 |
| Magnesium stearate | 1.0 |
| (d) Capsule | mg/capsule |
| Compound X | 10 |
| Lactose Ph.Eur | 488.5 |
| Magnesium | 1.5 |
| (e) Injection I | (50 mg/ml) |
| Compound X | 5.0% w/v |
| 1 M Sodium hydroxide solution | 15.0% v/v |
| 0.1 M Hydrochloric acid | |
| (to adjust pH to 7.6) | |
| Polyethylene glycol 400 | 4.5% w/v |
| Water for injection to 100% | |
| (f) Injection II | (10 mg/ml) |
| Compound X | 1.0% w/v |
| Sodium phosphate BP | 3.6% w/v |
| 0.1 M Sodium hydroxide solution | 15.0% v/v |
| Water for injection to 100% | |
| (g) Injection III | (1 mg/ml, buffered to pH6) |
| Compound X | 0.1% w/v |
| Sodium phosphate BP | 2.26% w/v |
| Citric acid | 0.38% w/v |
| Polyethylene glycol 400 | 3.5% w/v |
| Water for injection to 100% | |
| (h) Aerosol I | mg/ml |
| Compound X | 10.0 |
| Sorbitan trioleate | 13.5 |
| Trichlorofluoromethane | 910.0 |
| Dichlorodifluoromethane | 490.0 |
| (i) Aerosol II | mg/ml |
| Compound X | 0.2 |
| Sorbitan trioleate | 0.27 |
| Trichlorofluoromethane | 70.0 |
| Dichlorodifluoromethane | 280.0 |
| Dichlorotetrafluoroethane | 1094.0 |
| (j) Aerosol III | mg/ml |
| Compound X | 2.5 |
| Sorbitan trioleate | 3.38 |
| Trichlorofluoromethane | 67.5 |
| Dichlorodifluoromethane | 1086.0 |
| Dichlorotetrafluoroethane | 191.6 |
| (k) Aerosol IV | mg/ml |
| Compound X | 2.5 |
| Soya lecithin | 2.7 |
| Trichlorofluoromethane | 67.5 |
| Dichlorodifluoromethane | 1086.0 |
| Dichlorotetrafluoroethane | 191.6 |
| (l) Ointment | ml |
| Compound X | 40 mg |
| Ethanol | 300 μl |
| Water | 300 μl |
| 1-Dodecylazacycloheptan-2-one | 50 μl |
| Propylene glycol | to 1 ml |

Note

The above formulations may be obtained by conventional procedures well known in the pharmaceutical art. The tablets (a)-(c) may be enteric coated by conventional means, for example to provide a coating of cellulose acetate phthalate. The aerosol formulations (h)-(k) may be used in conjunction with standard, metered dose aerosol dispensers, and the suspending agents sorbitan trioleate and soya lecithin may be replaced by an alternative suspending agent such as sorbitan monooleate, sorbitan sesquioleate, polysorbate 80, polyglycerol oleate or oleic acid.

### EXAMPLE 4

Real Time PCR, Primers and Probes

| | |
|---|---|
| Mouse ADAMTS-1, set 1 | |
| Forward primer | SEQ ID NO: 16 |
| Reverse primer | SEQ ID NO: 17 |
| Probe | SEQ ID NO: 18 |
| Mouse ADAMTS-1, set 2 | |
| Forward primer | SEQ ID NO: 19 |
| Reverse primer | SEQ ID NO: 20 |
| Probe | SEQ ID NO: 21 |
| Human ADAMTS-1 | |
| Forward primer | SEQ ID NO: 22 |
| Reverse primer | SEQ ID NO: 23 |
| Probe | SEQ ID NO: 24 |

### EXAMPLE 5

Immunohistochemistry

The following paraffin embedded human material were used. Fatty streak (type I) from a young male and intermediate/advanced aortic plaques (type M-M) were used (courtesy pathology department, Sahlgrenska Hospital). Coronary artery was from a female between the ages 40-85. The sections were stained with eosin and hematoxylin (Cook 1974) to get an overview of the structure and degree of atherosclerosis.

To study the presence of smooth muscle cells, a commercial mouse monoclonal antibody against α-actin was used at 1:50 (Cedarlane labs). Another commercial mouse monoclonal antibody against HAM-56 (Daco), was used at 1:50-1:100 dilution to identify macrophages and possibly foam cells. Two rabbit antibodies raised against the same sequence from human ADAMTS-1 spacer domain were evaluated. Both gave similar results. Preabsorption with ADAMTS-1 peptide was used as control.

The immunohistochemistry was performed in an immunostainer, Techmate, from Daco. The primary antibodies were incubated on the sections for 12 hours, 25 minutes, followed by washing steps in TRIS buffered saline (RBS). The secondary antibodies were donkey-anti-rabbit-biotin (Jackson labs) diluted 1:2500 for ADAMTS-1 and donkey-anti-mouse-biotin (Jackson Labs) diluted 1:1000 for HAM-56 and α-actin. The secondary antibodies were incubated on the sections for 1 hours, followed by washing steps. Blockage of the endogenous peroxidase activity was performed 3×2.5 minutes with a kit from Daco for BP-blockage. After additional washing steps, HRP was incubated on the sections for 30 minutes, washed and finally the antigen-antibody complex was visualized by an EAC chromogen kit supplied by Daco for 3×7 minutes. The sections were washed, counterstained in hematoxylin, washed and mounted in Kaisers gelatin glycerine. All sections were examined in a Zeiss or an Olympus light microscope.

### EXAMPLE 6

In Situ Hybridization

Paraffin imbedded aorta from ApoE/LDL Receptor-deficient mice were used for the present study. No lesion was present in the tissue examined. A 35-S radiolabelled 500 base pair riboprobe was generated against the mouse ADAMTS-1 and used for the present study.

### EXAMPLE 7

Digestion of Proteoglycans

Human aortic smooth muscle cells were purchased from Clonetics (BioWhittaker) and cultured according to supplier. For preparation of total proteoglycan population (total PG), AoSMCs were seeded at 3000 cells/cm2 in SmBM2 media (4×80 cm2 flasks). 5 days later, the cells were washed with Dulbecco's PBS and BME-Diploid medium with FBS was added to the cells. 1 day later, fresh DME-Diploid medium without FBS, containing 35S-Sulfate 33 μCi/mL and 3H-Leucine 17 μCi/mL, 15 mL/bottle and incubated for 3 days. The medium was transferred and dialyzed against binding buffer containing 8M urea, 2 mM EDTA, 0.5% Triton X-100, and 20 mM Tris-HCl, pH 7.5, for 48 hours and applied to a pre-equilibrated Hi-Trap Q column. After washing with 25 mL of Elution buffer A (binding buffer+0.25 M NaCl, proteoglycan population is eluted with a linear salt gradient: 0.25-3 M NaCl in binding buffer. Total counts in each fraction were counted by liquid scintillation counting. The fractions containing PGs were pooled, dialyzed against water, lyophilized and stored at −20 degrees C. until use (‘total PG’).

Separation by size exclusion chromatography was performed using a Superdex 200 HR 10/30 from Amersham Pharmacia biotech. Total PG sample was dissolved in 50 mM Tris pH 7.5, 4 M Guanidinium-HCL and loaded on a pre-equilibrated column with flow at 0.5 mL/min. 1 mL fractions were collected and 35S measured for each fraction.

### EXAMPLE 8

Expression of hADAMTS-1 in THP-1 Cells

THP-1 cells were cultured in RPMI 1640 media supplemented with 10% FBS, penicillin-Streptomycin, sodium pyruvate, and nonessential amino acids (Sigma). PMA (Sigma) was dissolved in ethanol and diluted in the medium at a final concentration of 160 nM. LDL was equilibrated in PBS and diluted to 400 μg/mL and oxidized with 10 μM CuSO4 for 2.5 hours at 37 degrees C. The mildly oxidized LDL was equilibrated in RPMI 1640 and filter sterilized.

THP-1 cells were cultured in media containing PMA for 0, 2, 4, 8, and 8+1 days. Another set of plates were incubated with PMA for 8+1 days, with the mildly oxidized LDL also present in the last day. RNA was extracted and ADAMTS-1 message was analyzed by real time PCR.

### EXAMPLE 9

Assay Development

The complete mouse ADAMTS-1 cDNA has been cloned from epididymal fat tissue and inserted in mammalian expression vectors (for both constitutive and inducible expression). The protein is expressed in its native form. The human ADAMTS-1 homologue has been cloned and expressed in both its native form and with a cleavable epitope tag (his6) in the C-terminus in order to simplify detection and purification. Antibodies against various functional domains in both the mouse and human ADAMTS-1 molecule have been generated.

Examples of Suitable Assays:

ADAMTS-1 protease activity (cell based or using purified recombinant protein)

ADAMTS-1 activation (cell based or using purified recombinant protein)

Activation of ADAMTS-1 transcription in suitable cell lines

Disintegrin assay for monitoring ADAMTS-1/(target) protein interactions

Selection of peptide substrate for high-throughput screening. Full length or recombinant metalloprotease domain can be used to screen compounds. Currently, we have a 38 amino acid peptide, derived from the published cleavage site on the proteoglycan aggrecan, which can be used as a substrate suitable for high-throughput screening. Cleavage of the peptide by recombinant ADAMTS-1 has been confirmed by HPLC analysis. The peptide can be labelled with a fluorescence marker to screen by a FRET/quench-type assay. Alternatively, the peptide can be labelled at one end and immobilized to plates or beads at the other end, and cleavage can be monitored by release of labelled cleavage product. Peptide sequence: TSELVEGVTEPTVSQE\hat{ }LGQRPPVTYTPQLFESSGEASC, SEQ ID NO 25: (\hat{ } denotes cleavage site by ADAMTS-1)

Assays for pro-domain cleavage/ADAMTS-1 activation

An cell migration assay to measure the activity of ADAMTS-1, such as migration of aortic smooth muscle cells across a matrix-coated filter, is being established. The effect of exogenous recombinant ADAMTS-1 will be tested to determine whether higher levels of the protease can affect migration. When compounds become available, they will be tested to determine if reduced protease activity can affect activity.

### EXAMPLE 10

ADAMTS-1: Role of the Protease in Atherosclerosis

In the human aorta, ADAMTS-1 is expressed normally at levels barely detectable by immunohistochemistry in the media but at substantially higher levels in foam-like and smooth muscle cells of early fatty streaks and in the matrix-like core at the base of type III-IV lesions (FIGS. 6,7).). Staining with ADAMTS-1 antibodies co-localize with smooth muscle cell (□-actin) staining (FIGS. 6a, d and 7a, d). In early fatty streak, ADAMTS-1 staining also co-localizes with staining observed with the macrophage marker, HAM-56 (FIGS. 6a, c). Preabsorption with peptides used to generate the antibodies removed most of the staining with the ADAMTS-1 antibodies (FIGS. 6b, 7b). It has also been observed that ADAMTS-1 message is up-regulated substantially in human umbilical vein endothelial cells and cardiac microvascular endothelial cells under shear stress, suggesting a potential role in flow-dependent vascular remodelling (Bongrazio et al., 2000). In addition, ADAMTS-1 is detected in the aortic plaques of LDL Receptor/ApoE-deficient mice. In situ hybridization experiments suggest that ADAMTS-1 message is normally expressed by both aortic medial (smooth muscle layer) and endothelial cells of LDL Receptor/ApoE-deficient mice, suggesting that both cell types are capable of producing ADAMTS-1 FIG. 8). The message levels are low; however, staining was performed on sections of aorta without lesions. In experiments with the human monocyte/macrophage-like cell line (THP-1 cells), ADAMTS-1 message is induced with PMA, a reagent known to induced maturation of monocytes into macrophages, as measured by real time PCR (FIG. 9). Exposure to mildly oxidized LDL did not significantly change the level of ADAMTS-1 message.

ADAMTS-1 is able to cleave aggrecan, a proteoglycan containing GAG moieties (chondroitin sulfate); deletion experiments suggest that binding to the chondroitin sulfate domain is required for cleavage of aggrecan (Iozzo, 1998; Kuno et al., 2000; Schwartz et al., 1999). In addition, ADAMTS-1 is able to cleave another proteoglycan belonging to the same gene family, versican, which is expressed at high levels primarily by VSMC in atherosclerotic lesions (Evanko et al., 1998; Sandy et al., 2001). See FIG. 10. Total proteoglycan was isolated from primary aortic smooth muscle cells and incubated with or without ADAMTS-1 prior to separation by size exclusion chromatography. In the presence of ADAMTS-1, the size of the large proteoglycan population made up primarily of versican is reduced and the size of the peak corresponding to smaller sized proteoglycan population is increased, indicating that ADAMTS-1 was able to cleave and reduce the size of versican.

The localization of ADAMTS-1 at the base of type III-IV plaques, adjacent to the medial layer, suggests that ADAMTS-1 may play a role in promoting migration of VSMCs from the vessel wall to the lesions. Real time PCR (Taqman) analysis for ADAMTS-1 message indicates that it is expressed at a higher level in proliferating primary aortic VSMC compared to confluent cells in vitro, consistent with its potential role in promoting SMC migration in atherosclerosis (FIG. 11). Interestingly, parathyroid hormone-related protein, a hormone expressed by both arterial smooth muscle and endothelial cells and known to be mitogenic when targeted to the nucleus, can induced expression of ADAMTS-1 in bone (Miles et al., 2000; Massfelder et al., 1997). A C. elegans member of the ADAMTS family, gon-1, has been shown to play an essential role in the migration of distal tip cells during gonad morphogenesis, presumably by modifying basement membrane components (Blellock et al., 1999; Blellock and Kimble, 1999). Since a wild-type transgene could rescue the mutant phenotype while a protease-defective mutant could not, protease activity is essential for normal distal tip cell migration and gonad development. Although there is no aggrecan or versican ortholog in C. elegans, there are a number of uncharacterized chondroitin sulfate proteoglycans that may be substrates for GON-1. In addition, both aggrecan and versican has been shown to have a role in avian neural crest migration (Perissinotto et al., 2000). We have generated mice overexpressing the transgene for ADAMTS-1 as part of plans to further investigate the role of ADAMTS-1 in atherogenesis.

In a cross section through a normal aortic vessel, distinct layers of cells and matrix are observed. Versican binds other ECM proteins, such as tenascin and hyaluronic acid, and cell surface glycoproteins (e.g. CDA4-like protein detected on VSMC) and may form a hydrated aggregate which is expandable but resilient, much like aggrecan in cartilage (Hurt-Camejo, 1999). Although VSMC may require this proteoglycan/ECM-rich environment, the latter may also act as a physical barrier that prevents movement. In a normal aorta or vessel, VSMCs prefer to remain in the well delineated medial layer, in the diseased tissue, however, they migrate into the intima. ADAMTS-1 may be involved in making the intima more ‘permissive’ for invasion by the VSMCs. In addition, cleavage of proteoglycans may lead to the release of growth factors and cytokines to promote SMC migration from the media to the intima.

### REFERENCES

Blellock, R., Anna-Arriola, SS., Gao, D., Li, Y., Hodgkin, J. and Kimble, J. (1999). The gon-1 gene is required for gonadal morphogenesis in Caenorhabditis elegans. Developmental biology 216: 382-393.

Blellock, R. and Kimble, J. (1999). Control of organ shape by a secreted metalloprotease in the nematode Caenorgabditis elegans. Nature 399: 586-590.

Blobel, C P. (1997). Metalloprotease-Disintegrins: Links to cell adhesion and cleavage of TNF α and Notch. Cell 90, 589-592.

Bongrazio, M., Baumann, C., Zakrzewicz, A., Pries, A R., Gaehtgens, P. (2000). Evidence for modulation of genes involved in vascular adaptation by prolonged exposure of endothelial cells to shear stress. Cardiovascular research 47, 384-393.

Evanko, S P., Angello, J C., Wight, T N (1998). Formation of hyaluronan- and versican-rich pericellular matrix is required for proliferation and migration of vascular smooth muscle cells. Arterioscler. Thromb. Vasc. Biol. 19: 1004-1013.

Hurt-Camejo, E., Rosengren, B., Sartipy, P., Elfsberg, K., Cajemo, G., Svensson, L. (1999). CD44, a cell surface chondroitin sulfate proteoglycan, mediates binding of interferon-g and some of its biological effects on human vascular smooth muscle cells. JBC 274: 18597-18964.

Iozzo, R V. (1998). Matrix proteoglycans: From molecular design to cellular function. Annu. Rev. Biochem. 67: 609-52.

Kuno, K., Kanada, N., Nakashima, E., Fujiki, F., Ichimura, F., Matsushima, K (1997). Molecular cloning of a gene encoding a new type of metalloproteinase-disintegrin family protein with thrombospondin motifs as an inflammation associated gene. J. Biol. Chem. 272, 556-562.

Kuno, K. and Matsushima, K (1998). ADAMTS-1 protein anchors at the extracellular matrix through the thrombospondin type 1 motifs and its spacing region. J. Biol. Chem. 273, 13912-13917.

Kuno, K., Okada, Y., Kawashima, H., Nakamura, H., Miyasaka, M., Ohno, H., Matsushima, K. (2000). ADAMTS-1 cleaves a cartilage proteoglycan, Aggrecan. FEBS Letters 478: 241-245.

Liang, P. and Pardee, A B. (1992) Differential display of eukaryotic messenger RNA by means of the polymerase chain reaction. Science 257:967-971.

Massfelder, T., Dann, P., Wu, T L., Vasavada, R., Helwig, J J., Stewart, AF (1997). Opposing mitogenic and anti-mitogenic actions of parathyroid hormone-related protein in vascular smooth muscle cells: A critical role for nuclear targeting. PNAS 94:13630-13635.

Miles, R R, Sluka, J P, Halladay, D L., Santerre, R F., Hale, L B., Thirunavukkarasu, K, Galvin, R J S., Hock, J M., Onyia, J E. (2000). ADAMTS-1: A cellular disintegrin and metalloprotease with thrombospondin Motifs is a target for parathyroid hormone in bone. Endocrinology 141:4533-4542.

Perissinotto, D., Iacopetti, P., Bellina, L, Doliana, R., Colombatti, A., Pettway, Z., Bronner-Fraser, M., Shinomura, T., Kimata, K, Mörgelin, M., Löfberg, J., Periis, R. (2000). Avian neural crest migration is diversely regulated by the two major hyaluronan-binding proteoglycans PG-M/versican and aggrecan. Development 127: 2823-2842.

Primakoff, P. and Myles, D G. (2000). The ADAM gene family. Trends in Genetics 16, 83-87.

Sandy, J D., Westling, J., Kenagy, R D., Iruela-Arispe, U L., Verscharen, C., Rodriguez-Mazaneque, J C., Zimmermann, D R., Lemire, J M., Fischer, J W., Wight, T N., Clowes, A W. (2001). Versican V1 proteolysis in human aorta in vivo occurs at the glu441-ala442 bond, a site that is cleaved by recombinant ADAMTS-1 and ADAMTS4. JBC 276: 13372-13378.

Shindo, T., Kurihara, X, Kuno, K., Yokohama, H., Wada T., Kurihara, Y., Imai Y., Ogata, M., Nishimatsu, H., Moriyama, N., Oh-hashi, Y., Morita, H., Ishikawa, T., Nagai, R., Yazaki, Y. and Matsushima, K. (2000) ADAMTS-1: a metalloproteinase-disintegrin essential for normal growth, fertility, and organ morpholoy and function. J. Clin. Invest. 105, 1345-1352.

Schwartz, B N., Pirok, E W., Mensch, J R. and Domowicz, M S. (1999). Domain organization, genomic structure, evolution, and regulation of expression of the aggrecan gene family. Prog. Nuc. Acid. Res. 62: 177-225.

Tang, B L. and Hong, W. (1999). ADAMTS: A novel family of proteases with an ADAM protease domain and thrombospondin 1 repeats. FEBS letters 445, 223-225.

Tang, B L (2001). ADAMTS: A novel family of extracellular matrix proteases. Int. J. Biochem. Cell Biol. 33(1): 3344.

Wolfsberg, T G, Primakoff, P., Myles, D G., White, J M. (1995). ADAM, a novel family of membrane proteins containing A Disintegrin And Metalloprotease domain: Multipotential functions in cell-cell and cell-matrix interactions. J. Cell. Biol. 131, 275-278.

Wolfsberg, T G. and White, J M. (1996). ADAMs in fertilization and development. Developmental Biology 180, 389-401.

1. UseA method of treating a disease independently selected from obesity, IRS, NIDDM or atherosclerosis, comprising administering to a patient a therapeutic amount of a compound able to modulate specifically the activity or amount of ADAMTS-1 in preparation of a medicament for the treatment of a disease independently selected from obesity, IRS, NIDDM or atherosclerosis.

2. A usemethod according to claim 1, comprising administering to a patient a therapeutic amount of a compound able to reduce specifically the activity or amount of ADAMTS-1 in preparation of a medicament for the treatment of obesity, IRS, NIDDM or atheroscleosis.

3. A usemethod according to claim 1, comprising administering to a patient a therapeutic amount of a compound able to increase specifically the activity or amount of ADAMTS-1 in preparation of a medicament for the treatment of obesity, IRS, NIDDM or atherosclerosis.

4. A usemethod according to claim 1, comprising administering to a patient a therapeutic amount of a compound able to reduce specifically the activity of ADAMTS-1 in preparation of a medicament for the treatment of obesity, IRS, NIDDM or atherosclerosis.

5. A usemethod according to claim 1, comprising administering to a patient a therapeutic amount of a compound able to increase specifically the activity of ADAMTS-1 in preparation of a medicament for the treatment of obesity, IRS, NIDDM or atherosclerosis.

6. A method of screening for a compound potentially useful for treatment of obesity, IRS, NIDDM or atherosclerosis which comprises assay of the compound for its ability to modulate specifically the activity or amount of ADAMTS-1.

7. A method according to claim 6 in which the assay is independently selected from:
i) measurement of ADAMTS-1 activity using a cell line which expresses ADAMTS-1 or using purified ADAMTS-1 protein; and
ii) measurement of ADAMTS-1 transcription or translation in a cell line expressing ADAMTS-1.

8. A method according to claim 7 in which the cell line is a mouse 3T3-L1 cell.

9. A method according to claim 7 in which the protein is human recombinant ADAMTS-1.

10. A method of preparing a pharmaceutical composition which which comprises:
i) identifying a compound as useful for treatment of obesity, IRS, NIDDM or atherosclerosis according to a method of any one of claims 6-9;
ii) mixing the compound or a pharmaceutically acceptable salt thereof with a pharmaceutically acceptable excipient or diluent. cceptable excipient or diluent.

11. A method of preparing a pharmaceutical composition which which comprises:
i) identifying a compound as useful for treatment of obesity, IRS, NIDDM or atherosclerosis according to a method of claim 7;
ii) mixing the compound or a pharmaceutically acceptable salt thereof with a pharmaceutically acceptable excipient or diluent.

12. A method of preparing a pharmaceutical composition which which comprises:
i) identifying a compound as useful for treatment of obesity, IRS, NIDDM or atherosclerosis according to a method of claim 8;
ii) mixing the compound or a pharmaceutically-acceptable salt thereof with a pharmaceutically acceptable excipient or diluent.

13. A method of preparing a pharmaceutical composition which which comprises:
i) identifying a compound as useful for treatment of obesity, IRS, NIDDM or atherosclerosis according to a method of claim 9;
ii) mixing the compound or a pharmaceutically acceptable salt thereof with a pharmaceutically acceptable excipient or diluent.
